(19) United States
(12) Patent Application Publication
(10) Pub. No.: US 2014/0193840 A1
(43) Pub. Date: Jul. 10, 2014

(54) CHROMATOGRAPHY FILTER PAPER-BASED ELISA

(71) Applicant: NATIONAL TSING HUA UNIVERSITY, Hsinchu (TW)

(72) Inventors: Min-Yen HSU, Hsinchu (TW); Keng-Hung LIN, Hsinchu (TW); Chao-Min CHENG, Hsinchu (TW)

(73) Assignee: National Tsing Hua University, Hsinchu (TW)

(21) Appl. No.: 13/867,409

(22) Filed: Apr. 22, 2013

(30) Foreign Application Priority Data
Jan. 8, 2013 (TW) 102100580

(51) Int. Cl. G01N 33/74

(52) U.S. Cl. CPC G01N 33/74
USPC 435/7.92

(57) ABSTRACT

A chromatography filter paper-based ELISA includes following steps: providing a chromatography filter paper plate provided with a plurality of independent hydrophilic regions defined by at least one hydrophobic region; immobilizing an antigen of a sample onto one of the hydrophilic regions of the chromatography filter paper plate; and detecting the antigen with an ELISA process, wherein an antibody used in the ELISA process is a monoclonal antibody. The present invention has advantages in lower amount of loaded sample, higher sensitivity and rapid analysis in comparison to conventional ELISA.

### BACKGROUND OF THE INVENTION

1. Field of the Invention

The present invention relates to a chromatography filter paper-based ELISA and more particularly to a chromatography filter paper-based ELISA for lesion analysis of ocular angiogenesis.

2. Description of the Prior Art

As society grows wealthier and life expectancy of human increases, diseases of civilization such as diabetic retinopathy and macular degeneration become threats to humans' health. According to statistics, there are approximately more than one million of people suffering from macular degeneration in the U.S.A. These two diseases have been proved highly relevant to concentration of vascular endothelial growth factor (VEGF) in eyes. When the concentration of VEGF increases, lesions of diabetic retinopathy or macular degeneration become worse and may even lead to blindness.

In order to cope with these diseases, at least two types of antibody drugs are developed against VEGF. The older version is Bevacizumab (Avastin) and the newer one is ranibizumab (lucentis), wherein the latter one is modified based on the older version and provided with less molecular weight. Both of these drugs are used to reduce the concentration of VEGF in eyes so as to reduce vascular proliferation and prevent deterioration in vision.

However, due to complexity and specialization of the nervous structure of the eyes, general blood testing is not applicable to assess lesion activity of eye diseases. Besides, there is no suitable method to sample eyes fluid for analysis of the growth factors in lesions. Therefore, ocular lesions become a conundrum to be solved since the VEGF concentration cannot be traced by molecular testing so far.

Enzyme-linked immunosorbent assay (ELISA) is a test to detect the presence of a substance and has been used in various industries. Based on the special binding properties between antigens and antibodies, the specific antibodies added can bind to the antigens of the sample. Subsequently, a substrate of the enzyme is added and displays color change. Based on colorimetric reaction of the enzyme, it can be determined whether the antigens bind with the antibodies for qualitative analysis. Additionally, the degree of combination of the antigens and antibodies may yield color intensity for quantitative analysis.

Traditionally, a 96-well microplate (manufactured by plastic injection molding) is used for ELISA. It is adequate for quantitative analysis and high-throughput screening. Nevertheless, analyte and reagents of each test requires relatively large volume (approx. 20˜200 μl) and long time for operation. It still needs to be improved.

Dot-immunobinding assays (DIA) uses porous membranes of nitrocellulose filter paper. Although DIA allows simple immunoassay performed on the paper, each test consumes a new piece of nitrocellulose filter paper and needs to be treated individually in the petri dish for few hours. Currently, most DIA are considered qualitative tests but not quantitative tests, which only provides Yes/No results.

U.S. Pat. No. 7,083,912 discloses a Dot-ELISA for detecting viruses. It uses nitrocellulose and monoclonal antibodies to promote detection sensibility. Mainly, it contributes to qualitative detection but not quantitative detection.

In brief, it is a difficult challenge to monitor VEGF concentration from the anterior chamber of eyeballs according to present techniques. The maximum amount of aqueous humor which can be extracted from the anterior chamber of eyeballs is 0.2 mL and each well of a 96-well microplate requires around 0.1 mL of aqueous humor. As a result, a single traditional ELISA test (maximum for two times) can be performed once for each patient and cannot be repeated for many times to obtain more precise clinical information.

Hence, there remains a need in the art to develop a rapid analysis provided with higher sensibility and only requiring lower amount of loaded sample for ELISA.

### SUMMARY OF THE INVENTION

It is an object of the present invention to provide a chromatography filter paper-based ELISA, which has advantages in lower amount of loaded sample, higher sensitivity and rapid analysis.

According to one embodiment of the present invention, a chromatography filter paper-based ELISA, comprising: providing a chromatography filter paper plate having a plurality of independent hydrophilic regions defined by at least one hydrophobic region; immobilizing an antigen of a sample onto one of the hydrophilic regions of the chromatography filter paper plate; and detecting the antigen with an ELISA, wherein a monoclonal antibody is used in the ELISA.

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

FIG. 1 is a flowchart schematically illustrating the steps of the chromatography filter paper-based ELISA of the present invention;

FIG. 2 is a schematic diagram illustrating a chromatography filter paper plate according to an embodiment of the present invention;

FIGS. 3A and 3B are diagrams of the experimental data displaying sensitivity of the chromatography filter paper-based ELISA according to an embodiment of the present invention;

FIG. 4A to FIG. 4E are photos displaying ocular vascular lesions of diabetic retinopathy patients;

FIG. 5A to 5F are photos displaying ocular vascular lesions of age-related macular degeneration patients;

FIG. 6A to 6F are photos displaying ocular vascular lesions of retinal vein occlusion patients; and

FIG. 7A to 7B are photos displaying ocular vascular lesions of age-related macular degeneration patients before and after treatments.

### DESCRIPTION OF THE PREFERRED EMBODIMENT

Enzyme-linked immunosorbent assay (ELISA) detects biological samples based on specific binding properties between the antigens and antibodies. Because the antigens or antibodies still have immune activity after binding with solid-phase substance, existence of the specific antigens or antibodies can be detected according to colorimetric reaction of enzymes under specific binding mechanism.

A chromatography filter paper-based ELISA according to the present invention (hereinafter referred as paper-based ELISA) allows qualitative and quantitative analysis and is free from the problem of conventional DIA (only for qualitative analysis). According to the present invention, an “indirect ELISA” is herein applied, namely the antigens are immobilized onto the paper plate to be detected and then the ELISA is applied to determine the amount of the monoclonal antibodies so as to detect the antigen concentration.

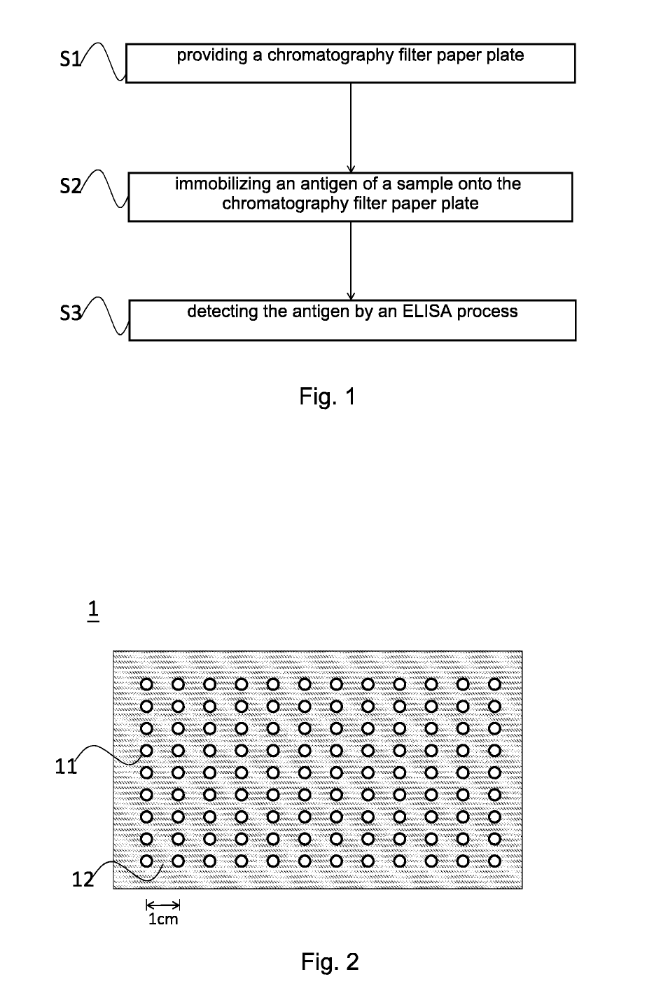

Referring to FIG. 1 and FIG. 2, in step S1, a chromatography filter paper-based ELISA according to the present invention uses a chromatography filter paper plate 1 having a plurality of independent hydrophilic regions 11 defined by at least one hydrophobic region 12. The hydrophilic regions 11 are surrounded and respectively isolated by the hydrophobic region 12. Size, amount and shape of the hydrophilic regions 11 are not restricted. As shown in the figure, in an embodiment, configuration of a conventional 96-well microplate is adopted for the paper-based ELISA according to one embodiment of the present invention. Shape and size of each well is uniform with the line spacing of approximate 1 cm. Moreover, 24-well or 384-well microplates also can be used. As can be seen, arrangement of the hydrophilic regions 11 is not restricted and can be customized according to different requirements.

The chromatography filter paper of the present invention is semipermeable and used for phase separation. For example. It can separate solid substance from liquid or air. Materials of filter papers are plant fibers which are usually derived from lumbers and cottons. In one preferred embodiment, the chromatography filter papers are Whatman® cellulose chromatography filter papers available in the market, which are made of cotton fibers.

The chromatography filter paper of the present invention is distinguished from the conventional DIA nitrocellulose filter paper, especially in adsorption properties. After dropping liquid glucose on the surface of a chromatography filter paper and a nitrocellulose filter paper, absorption properties can be observed that liquid glucose remains on the surface of nitrocellulose filter paper and can hardly percolate therethrough, which is similar to its surface absorption properties during protein transferring. In contrast, the chromatography filter papers of the present invention have better water permeability and are able to absorb more solvents. Obviously, the chromatography filter papers of the present invention are believed to achieve better results than nitrocellulose filter papers.

People skilled in the art should understand different ways for defining the hydrophobic region 12 of the chromatography filter paper plate 1. For instance, in one preferred embodiment of the present invention, the hydrophobic region 12 is formed by coating chemical materials on the paper plate such as wax printing. Processes of wax printing are reported by Carrilho, E et al. (“Understanding wax printing: a simple micropatterning process for paper-based microfluidics”, Anal Chem, 81, 7091-7095, 2009)

In one embodiment, specific patterns can be printed on the chromatography filter paper by wax printing. After that, the patterned chromatography filter paper is baked (100° C., 10 min) to obtain the chromatography filter paper plate 1 of the present invention.

People skilled in the art should understand there are different methods for preparing the chromatography filter paper plate 1. In one embodiment, after coating a photoresist layer thereon and followed by UV irradiation, the hydrophobic region 12 can be formed to define the hydrophilic regions 11.

In step S2, an antigen of a sample is immobilized onto one of the hydrophilic regions 11. The most convenient manner is using the sample to moisten the hydrophilic regions 11 and then dehydrating or drying the hydrophilic regions 11 to finish immobilization.

Antigens are known as substance inducing immune response. In ELISA test, sorts of subjective antigens can be detected by antibodies. In other words, antigens which can be recognized by antibodies can become test subjects according to the present invention. Examples of antigens comprise growth factors, viruses, bacteria, tumor markers. Besides, an antibody which can be recognized by other antibodies is also adequate to be test subjects for the ELISA.

The aforementioned growth factors comprise but not limited to basic fibroblast growth factor (bFGF), vascular endothelial growth factor (VEGF), brain-derived neurotrophic factor (BDNF), transforming growth factor (TGF) or other growth factors.

Apart from this, in one embodiment, ELISA of the present invention can be applied to analyze the content of the analyte of the liposome or microspheres. By dispersing the analyte in liposome or microspheres as antigens and followed by the ELISA testing, the content of the analyte can be determined.

In step S3, a monoclonal antibody is used to bind with specific antigens. Subsequently, enzyme-linked immunosorbent assay which uses the monoclonal antibody is carried out to detect the antigens.

The amount of monoclonal antibodies can be determined by colorimetric reaction, fluorescence, luminescence, radiation or other signals. Conventional ELISA uses enzymes and reagents to induce colorimetric reaction so as to display presence of the antigens or analyte. Colorimetric enzymes comprise Horseradish peroxidase (HRP), Alkaline Phosphatase, beta-galactosidase, Glucose Oxidase or Glucose-6-phosphate Dehydrogenase. New types of ELISA comprise fluorescence, luminescence and real-time PCR reagents generating recognizable signals. From the aspect of technical classification, although new types of ELISA may have linking reaction between enzymes and non-enzyme reactants instead of original enzyme-linked reaction, these technical principles are basically similar to conventional ELISA and both belong to the same technical field. Accordingly, new types of ELISA are also included into the ELISA of the present invention.

In one embodiment, a colorimetric enzyme is conjugated to the monoclonal antibody to determine the amount of the monoclonal antibody via colorimetric reaction of the colorimetric enzyme, wherein the colorimetric enzyme may be a horseradish peroxidase (HRP). That is, by binding the monoclonal antibody with HRP, the amount of the monoclonal antibody can be determined via detecting the amount of HRP which binds with the monoclonal antibody. For HRP reaction system, 3,3′,5,5′-tetramethylbenzidine (TMB) can be provided as reactants along with streptavidin as blocking agents to increase sensibility of the reaction.

Additionally, in one embodiment, the amount of the monoclonal antibody can be determined by the second antibody which is used for specifically recognizing the monoclonal antibody. In another embodiment, the second antibody conjugated with HRP is used to perform the colorimetric reaction described above. The step using the second antibody for ELISA detection is commonly adopted by people skilled in the art and will not be elaborated herein.

In one embodiment, the present invention can be applied in detection or prognosis of ocular vascular lesions. Common lesions comprise retinal vein Occlusion (RVO), proliferative diabetic retinopathy (PDR), neovascular glaucoma (NVG), age-related macular degeneration (AMD) etc.

From the aspect of pathology, vascular proliferation in eyeballs usually leads to blindness. Vascular proliferation for many diseases usually results from angiogenic factors released during the course of disease. Angiogenic factors comprise but not limited to vascular endothelial growth factor (VEGF), fibroblast growth factor (FGF), platelet-derived endothelial growth factor (PDEGF) and angiopoetin. Vascular endothelial growth factors induce proliferation of vascular endothelial cells and further lead to formation of spacing within microvessel endothelial cells. As a consequence, a suitable circumstance for vascular proliferation is established owing to increasing permeability, fibrinogen loss and structural change of surrounding connective tissues.

By detecting vascular growth factors in eyeballs especially for detecting VEGF concentration of aqueous humor or vitreous humor, growing trends of vascular proliferation can be predicted so as to estimate how ocular lesions deteriorate. An intraocular chamber comprises a anterior chamber, a posterior chamber and a vitreous cavity. Intraocular elements include aqueous humor filling in the anterior chamber and vitreous humor filling in the posterior chamber, and vitreous. These three elements are transparent with certain refractive index and are incorporated with cornea into a refractive system.

Related to the monoclonal antibody used for detecting VEGF, there are currently two types of VEGF antibodies available in the market. One is the older version of Bevacizumab (also known as Avatin) and the other is Ranibizumab (also known as lucentis) based on modification of the previous one for less molecular weight. The drugs described above are both monoclonal antibodies and can be implemented on paper-based ELISA of the present invention.

The detailed explanation of the present invention is described as follows. The described preferred embodiments are presented for purposes of illustrations and description, and they are not intended to limit the scope of the present invention.

Using Chromatography Filter Paper-Based ELISA to Detect VEGF

According to the present invention, it first provides a chromatography filter paper plate. After moistening the paper plate, VEGF antigens are added thereon and stand for five to seven minutes. Then, bovine serum albumin (BSA) is added as blocking agents to prevent non-specific binding. After standing for 5 to 7 minutes, VEGF monoclonal antibodies which conjugate HRP are added to react with the antigens for 7 to 10 minutes. Next, the second blocking agent Streptavidin is added to react with reagents for 7 to 10 minutes. Finally, the chromatography filter paper plate is rinsed. Meanwhile, the solution comprising 3,3′,5,5′-tetramethylbenzidine (TMB) and H₂O₂ are also added thereon until dry. After capturing the images of the paper plate, the images can be analyzed for gaining information.

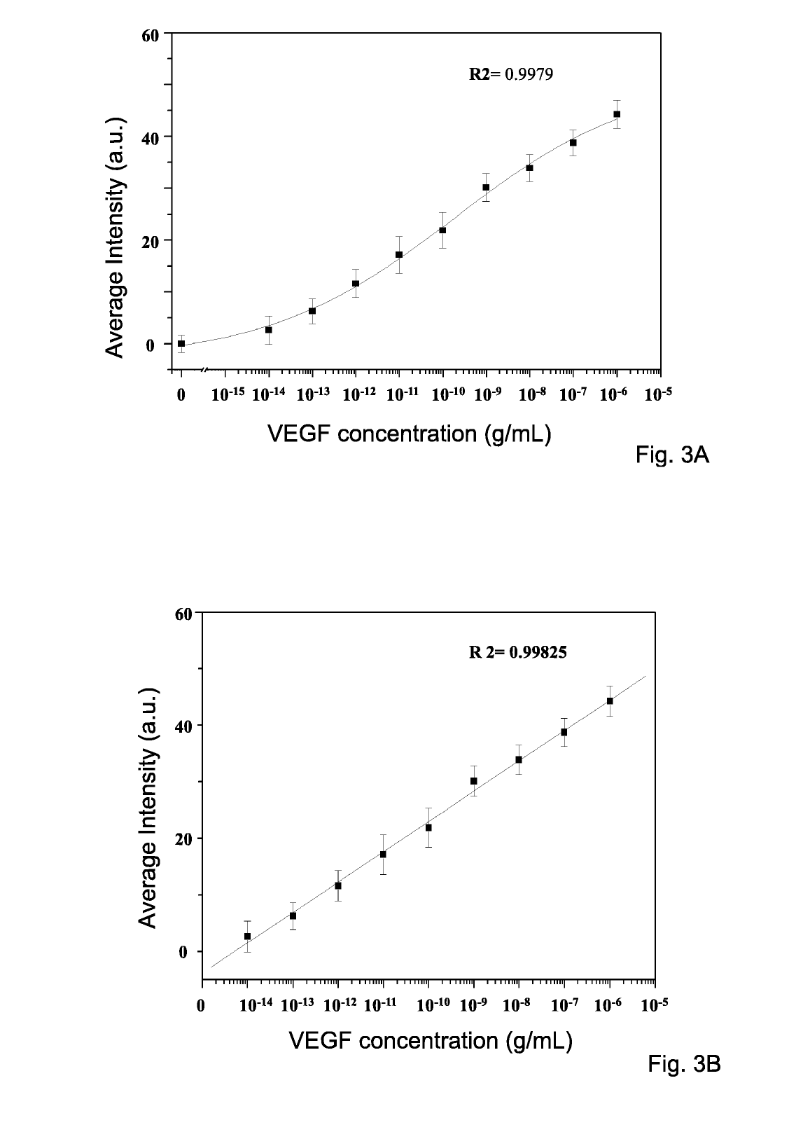

As displayed in FIGS. 3A and 3B, they are calibration curves illustrating logarithmic value of the VEGF concentration absorbed in each testing region based on average intensity of colorimetric reaction derived from HRP enzyme reacting in ELISA testing. Each point of the curve is the average value repeated for eight times (N=8) and error bars represents standard deviation of the detected results. After scanning the 96-well microplate paper plate, it uses PhotoShop® to analyze color intensity of each testing region and followed by calculating the correlating coefficient R2 of the curve via Hill Equation, wherein R2=0.9979 (FIG. 3A). Besides, the range of the curve between 10⁻¹⁴ and 10⁻⁶ g/mL can be linearly approximated, wherein the corresponding correlation coefficient R2 is 0.99825. (FIG. 3B).

TABLE 1
the VEGF value and concentration of diabetic retinopathy
patients, detected by ELISA of the present invention.
Diabetic retinopathy

| | | converting |
|---|---|---|
| | AVarage VEGF | concentration |
| Items | value | (pg/ml) |
| Patient A | 31.2514 | 2027.040 |
| Patient B | 25.323 | 256.669 |
| Patient C | 22.41333 | 97.336 |

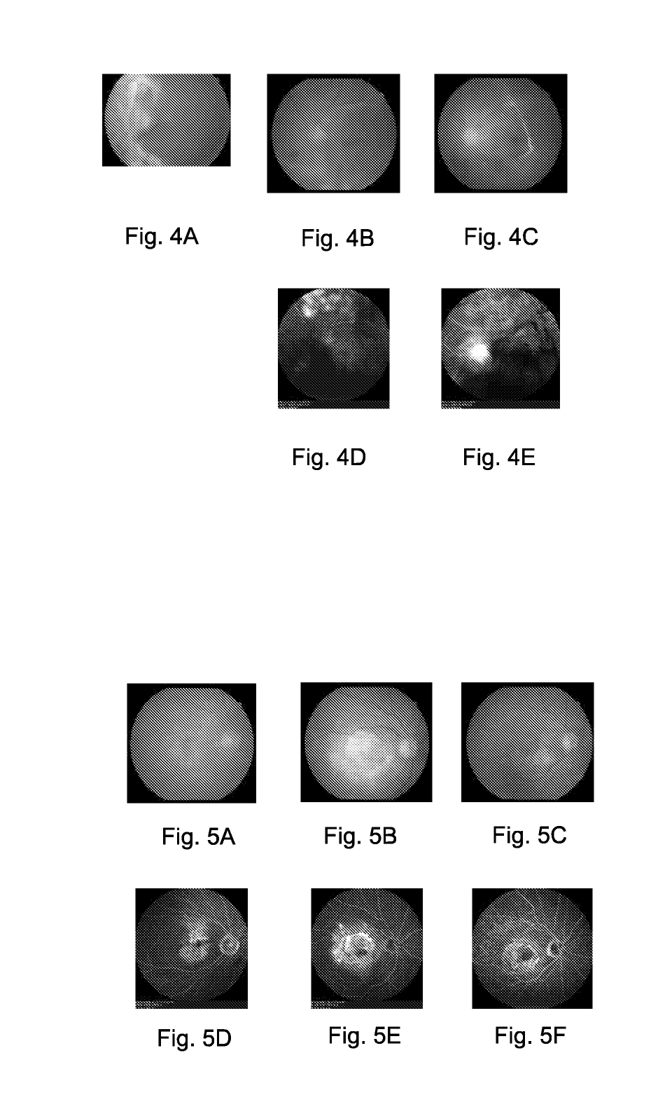

As described before, the paper-based ELISA of the present invention can be applied to detection or prognosis of ocular vascular lesions. Referring to Table 1 and FIG. 4A to 4E, they are images displaying diabetic retinopathy lesions. The average VEGF value of Patient A is 31.2514 and the converted concentration reading thereof is 2027.040 pg/ml. FIG. 4A is the photo displaying retina of Patient A. The average VEGF value of Patient B is 25.323 and the converted concentration reading thereof is 256.669 pg/ml. FIG. 4B is the photo displaying the retina of Patient B and FIG. 4D is the fluorescent photo displaying retinal vessels of Patient B. The average VEGF value of Patient C is 22.41333 and the converted concentration reading thereof is 97.336 pg/ml. FIG. 4C is the photo displaying the retina of Patient C and FIG. 4E is the fluorescent photo displaying the retinal vessels of Patient C.

TABLE 2
the VEGF value and concentration of age-related macular
degeneration patients, detected by ELISA of the present invention.
Age-related macular degeneration

| | | converting |
|---|---|---|
| | Avarage VEGF | concentration |
| Items | value | (pg/ml) |
| Patient D | 23.501 | 139.855 |
| Patient E | 26.124 | 335.773 |
| Patient F | 18.927 | 29.882 |

Referring to Table 2 and FIG. 5A to 5F, FIG. 5A to 5E are images displaying age-related macular degeneration lesions, wherein FIG. 5A is the photo displaying retina of Patient F and FIG. 5D is the fluorescent photo displaying the retinal vessels of Patient F. The average VEGF values and converted concentration reading of Patient D, E and F are listed in Table 2.

TABLE 3
the VEGF value and concentration of Retinal vein
occlusion patients, detected by ELISA of the present invention.
Retinal vein occlusion

| | | converting |
|---|---|---|
| | Avarage VEGF | concentration |
| Items | value | (pg/ml) |
| Patient G | 26.23 | 348.004 |
| Patient H | 28.87 | 860.023 |
| Patient I | 17.268 | 16.663 |

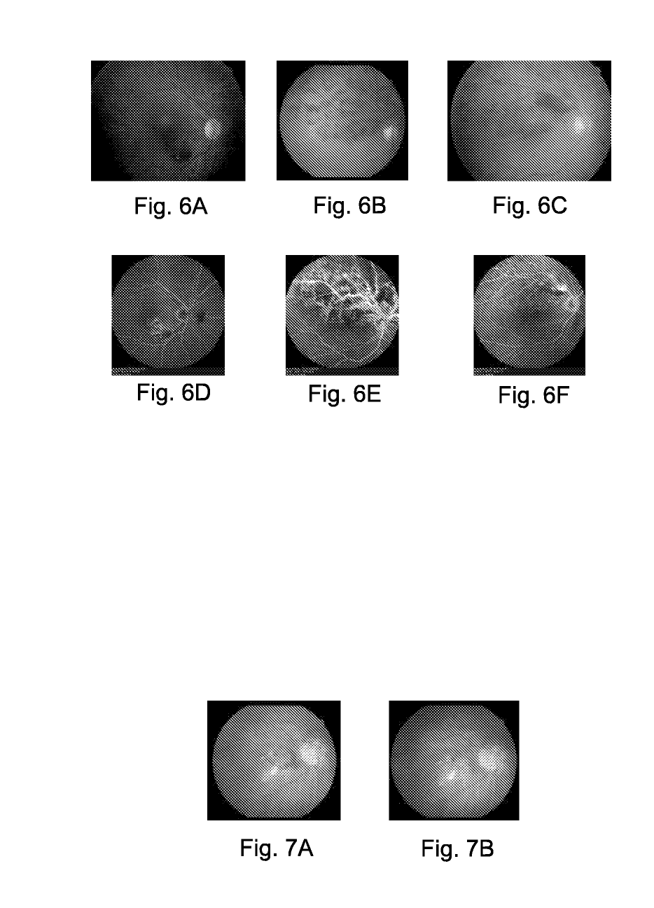

Referring to Table 3 and FIG. 6A to 6F, FIG. 6A to 6E are images displaying age-related macular degeneration lesions, wherein FIG. 6A is a photo displaying the pupils of Patient G and FIG. 6D is a fluorescent photo displaying the retinal vessels of Patient G; FIG. 6B is a photo displaying the retina of Patient H and FIG. 6E is a fluorescent photo displaying the retinal vessels of Patient H; FIG. 6C is a photo displaying the retina of Patient I and FIG. 6F is a fluorescent photo displaying the retinal vessels of Patient I. The average VEGF values and converted concentration reading of Patient G, H and I are listed in Table 3.

TABLE 4
the VEGF value and concentration of age-related
macular degeneration patients before and after treatment,
detected by ELISA of the present invention.

| | | converting |
|---|---|---|
| | Avarage VEGF | concentration |
| Items | value | (pg/ml) |
| Before treatment | 26.23 | 348.004 |
| After treatment | 28.87 | 860.023 |

Referring to Table 4, it shows the VEGF values and converted concentration reading of age-related macular degeneration patients before and after treatments, detected by ELISA of the present invention. Tissue fluid is extracted from an age-related macular degeneration patient aged 80 for paper-based ELISA. Results show the VEGF concentration is 93.762 pg/mL (average VEGF value=22.301). Referring to FIG. 7A and FIG. 7B, FIG. 7A is a image of retina showing that bleeding occurs near the macular region of the right eye. One week after injecting Bevabizmab into the vitreous of the right eye, the paper-based ELISA shows that the VEGF concentration is 20.740 pg/mL (average VEGF value=17.883). FIG. 7B is an image of retina showing that bleeding phenomenon near the right eye has been alleviated.

Comparing to previous technique, fifty times of complete test can be finished by the paper-based ELISA using 0.1 mL of aqueous humor in the eyeballs according to the present invention, which is more effective than the conventional manner which can only carry out 1/96 ELISA test. Efficiency ratio for amount of loaded sample in ELISA is around 4800:1. With respect to testing time, a complete test of paper-based ELISA only needs 40 min. In contrast, the conventional ELISA requires 213 min. Time efficiency ratio is around 5:1.

In conclusion, the present invention uses monoclonal antibodies to detect the concentration of vascular endothelial growth factor in aqueous humor so as to increase sensibility of ELISA. This novel ELISA can be applied as markers for clinical diagnosis, which are good reference for diagnosing and tracing disease activity. Specifically, it provides medical evidence for molecular diagnosis to diabetic retinopathy, retinal vein occlusion and age-related macular degeneration so that benefits diagnosing or prognosis of disease activity.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the spirit and scope of the appended claims.

What is claimed is:

1. A chromatography filter paper-based ELISA comprising:
providing a chromatography filter paper plate having a plurality of independent hydrophilic regions defined by at least one hydrophobic region;
immobilizing an antigen of a sample onto one of the hydrophilic regions of the chromatography filter paper plate; and
detecting the antigen by an ELISA process, wherein a monoclonal antibody is used in the ELISA process.

2. The chromatography filter paper-based ELISA according to claim 1, wherein the chromatography filter paper plate is made of cotton fibers.

3. The chromatography filter paper-based ELISA according to claim 1, wherein the hydrophobic region is formed by wax printing.

4. The chromatography filter paper-based ELISA according to claim 1, wherein the antigen is a growth factor.

5. The chromatography filter paper-based ELISA according to claim 1, wherein the sample is aqueous humor or vitreous humor.

6. The chromatography filter paper-based ELISA according to claim 1, wherein the antigen is an angiogenic factor.

7. The chromatography filter paper-based ELISA according to claim 6, wherein the angiogenic factor comprises vascular endothelial growth factor (VEGF), fibroblast growth factor (FGF), platelet-derived endothelial growth factor (PDEGF) or angiopoietin.

8. The chromatography filter paper-based ELISA according to claim 5, wherein the antigen is vascular endothelial growth factor (VEGF).

9. The chromatography filter paper-based ELISA according to claim 5, wherein the monoclonal antibody is Bevacizumab or Ranibizumab.

10. The chromatography filter paper-based ELISA according to claim 1, wherein the antigen is dispersed in a liposome or a microsphere.

11. The chromatography filter paper-based ELISA according to claim 1 further comprising:
determining the amount of the monoclonal antibody so as to detect the concentration of the antigen.

12. The chromatography filter paper-based ELISA according to claim 11, wherein a colorimetric enzyme conjugated to the monoclonal antibody is used to determine the amount of the monoclonal antibody via detecting a colorimetric reaction of the colorimetric enzyme.

13. The chromatography filter paper-based ELISA according to claim 11, wherein the colorimetric enzyme is a horseradish peroxidase (HRP).

14. The chromatography filter paper-based ELISA according to claim 11, wherein the colorimetric enzyme comprises alkaline phosphatase, beta-galactosidase, glucose oxidase or glucose-6-phosphate dehydrogenase.

15. The chromatography filter paper-based ELISA according to claim 11 further comprising:
using a second antibody to bind to the monoclonal antibody to determine the amount of the monoclonal antibody via detecting the amount of the second antibody.
